(19) United States
(12) Patent Application Publication
(10) Pub. No.: US 2004/0254610 A1
(43) Pub. Date: Dec. 16, 2004

(54) Miniature pocket electric stimulator

(76) Inventor: Hsin-Yi Lin, Shindian City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/864,315

(22) Filed: Jun. 10, 2004

(30) Foreign Application Priority Data
Jun. 13, 2003 (TW) 92210821

(51) Int. Cl. A61N 1/18

(52) U.S. Cl. 607/2; 607/46; 607/48

(57) ABSTRACT

A miniature pocket electric stimulator includes a case on which a first and a second seat are formed. The second seat is raised from the first seat and has a peripheral wall. A first electrode having an opening corresponding to the second seat is connected to the first seat. A second electrode is connected to the second seat via the opening on the first electrode, and is separated from the first electrode by the peripheral wall of the second seat. The first and second electrode-contacting elements mounted in the case electrically connect the first and the second electrode, respectively, to a circuit board, so that electric stimulating signals generated by a control circuit on the circuit board are sent to the first and the second electrode.

### BACKGROUND OF THE INVENTION

1. Field of the Invention

The preset invention relates to an electric stimulator, and more particularly to a miniature pocket electric stimulator.

2. Description of the Prior Art

Electric stimulators have been widely employed to electrically stimulate skin, nerves, and muscles at selected areas on human body for the purpose of rehabilitation and improving blood circulation. The electric stimulator has been proven in both western and Chinese theoretical and clinic medicines to have specific effects when an electric stimulating signal of proper intensity and frequency is applied to specific areas or points on a user's body.

In most conventional electric stimulating techniques, a main unit of the electric stimulator generates low-frequency pulse signals that are sent to two conductive patches via two conductors, and the conductive patches are then attached to selected points on the user's body to achieve the purpose of electrical stimulating.

The conventional electric stimulators are not structurally designed to enable convenient use thereof. To use a conventional electric stimulator, a user must connect the main unit of the electric stimulator to the two patches via a pair of conductors to achieve the function of electrical stimulating, making the conventional electric stimulator inconvenient for use and carrying.

Moreover, the conductive patches or electrodes of the conventional electric stimulators are normally applied at an outer surface with an adherent conductive material for attaching the electrodes to a user's skin. However, once the adherent material has degraded adhering effect, or become cured or dried, the electrodes would fail to provide the expected effect.

It is therefore desired to develop a miniaturized electric stimulator to improve the drawbacks existed in the conventional electric stimulators and to provide users with a better choice.

### SUMMARY OF THE INVENTION

A primary object of the present invention is to provide a pocket electric stimulator, with which a user may proceed with electrical stimulation at hands and other body areas.

Another object of the present invention is to provide a convenient miniature electric stimulator having a modularized structure. Conductors are omitted to enable convenient carrying and use by a user.

A further object of the present invention is to provide a convenient pocket electric stimulator that need not be applied with conductive paste. With novel arrangement of electrodes on the electric stimulator, the electric stimulator is particularly suitable for use as a pocket stimulator at any time without the need of applying conductive paste on the electrodes or externally connecting to conductive patches.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an assembled perspective view of a miniature pocket electric stimulator according to a preferred embodiment of the present invention;

FIG. 2 is an exploded top perspective view of the miniature pocket electric stimulator of FIG. 1;

FIG. 3 is an exploded bottom perspective view of the miniature pocket electric stimulator of FIG. 1;

FIG. 4 is a cross-sectional view taken along line 4-4 of FIG. 1; and

FIG. 5 is a perspective view of the miniature pocket electric stimulator of FIG. 1 having a loop connected to an end thereof.

### DETAILED DESCRIPTION OF THE PREFERRED EMBODIMENTS

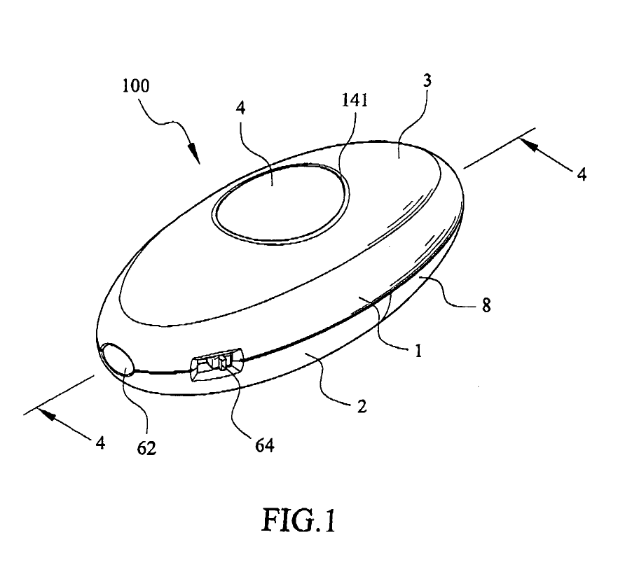

Please refer to FIGS. 1 to 3 in which a miniature pocket electric stimulator 100 according to a preferred embodiment of the present invention is shown. As shown, the electric stimulator 100 mainly includes an upper case 1 and a lower case 2 that are aligned with and connected to each other using generally known fastening means 21 and screws 55, 56 to define an internal space between them for receiving or mounting related components therein. In the illustrated embodiment, the electric stimulator 100 has a generally oblong configuration. However, it is understood the electric stimulator 100 may be produced to show many different appearances and configurations.

The upper case 1 includes a first seat 11 formed at a top thereof. A plurality of first retaining holes 12 are provided along a periphery of the first seat 11, and a first through hole 13 is provided on the first seat 11 at a predetermined position. A central portion of the first seat 11 is a raised platform to provide a second seat 14. A wall portion 141 is formed along a periphery of the second seat 14, a plurality of second retaining holes 15 are provided at the periphery of the second seat 14 close to an inner side of the wall portion 141, and a second through hole 16 is provided on the second seat 14 at a predetermined position.

The electric stimulator 100 includes a first electrode 3 that is one of two output electrodes of the present invention. The first electrode 3 has a configuration corresponding to the first seat 11 and the second seat 14 on the upper case 1. That is, the first electrode 3 is provided along a periphery with a plurality of first retaining hooks 31 adapted to align and engage with the first retaining holes 12, and at a central area with an opening 32 adapted to align and engage with the second seat 14. Whereby, the first electrode 3 may be firmly connected to the top of the upper case 1 through engagement of the first retaining hooks 31 with the first retaining holes 12, and the opening 32 with the wall portion 141 of the second seat 14.

A second electrode 4 that forms the other output electrode of the present invention is provided along a periphery with a plurality of second retaining hooks 41 for engaging with the second retaining holes 15 on the second seat 14 and thereby connecting the second electrode 4 to the second seat 14 via the opening 32 on the first electrode 3.

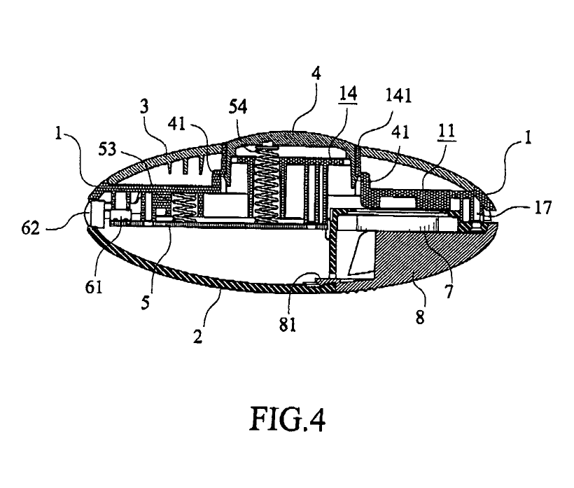

Please also refer to FIG. 4 that is a cross-sectional view of the electric stimulator 100 taken along line 4-4 of FIG. 1. When the second electrode 4 is connected to the second seat 14 via the mutually engaged second retaining hooks 41 and second retaining holes 15, it is fitly located in the opening 32 of the first electrode 3, but is separated from the first electrode 3 by the wall portion 141 of the second seat 14.

In the preferred embodiment of the present invention, both the first electrode 3 and the second electrode 4 are made of a plastic material, such as Alkyl Benzene Sulfonate (ABS), which is then electrically plated to form a plated conductive coating on an outer surface thereof. However, it is understood the first and the second electrode 3, 4 may also be made of other conductive materials, such as conductive rubber, aluminum, and other metallic materials.

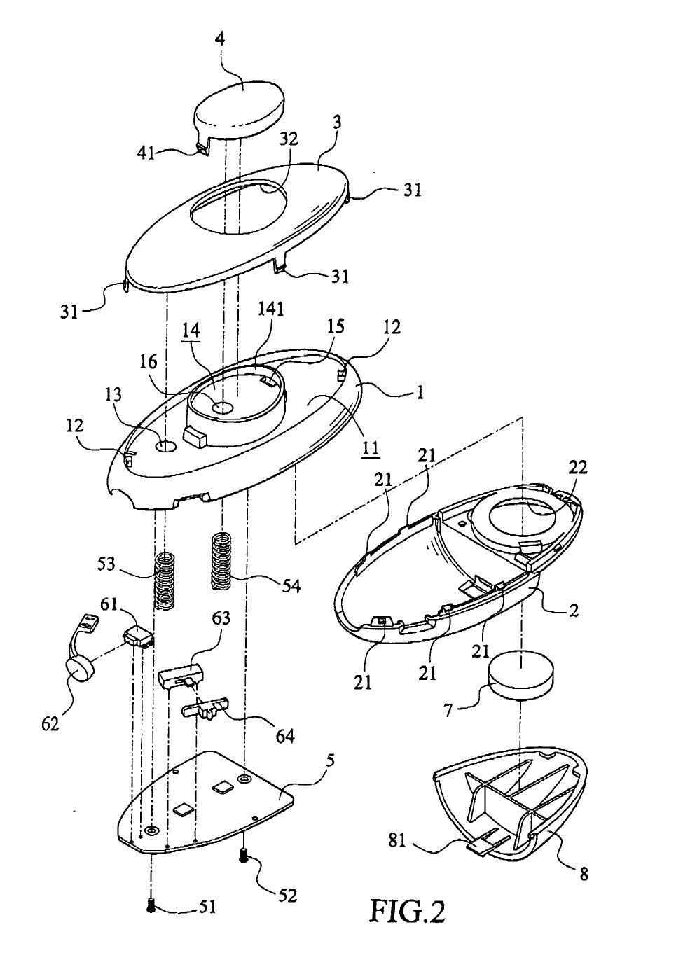

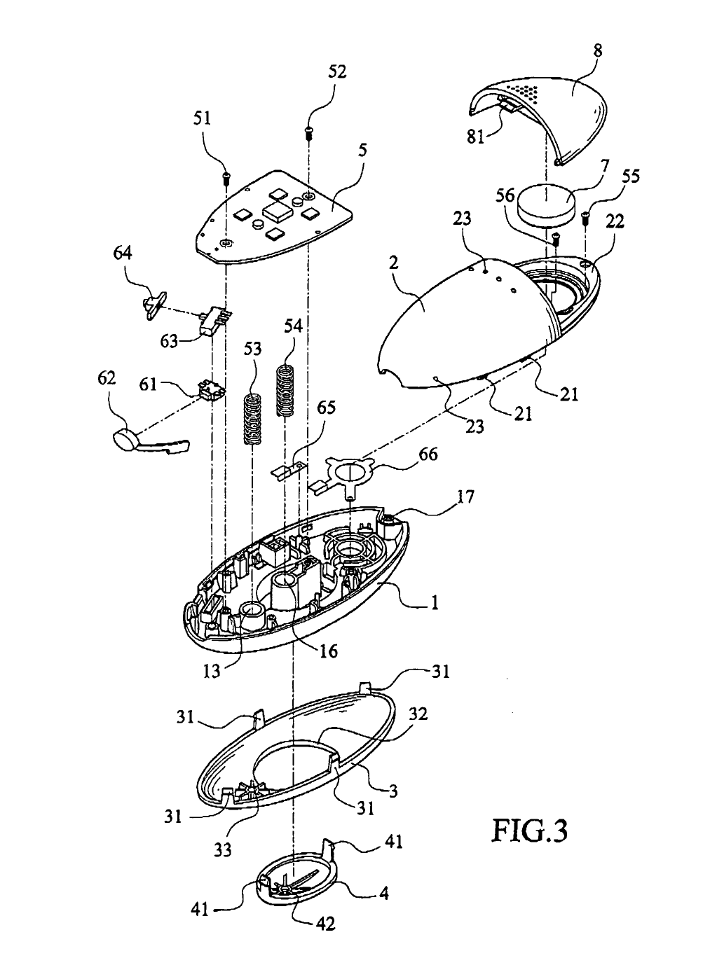

As can be seen from FIGS. 2 and 3, the upper and the lower case 1, 2 are connected together with screws 51, 52, 55, and 56, and a circuit board 5 is fixedly mounted between the upper and the lower case 1, 2 by the screws 51, 52. A first electrode-contacting element 53 and a second electrode-contacting element 54, which are two coil springs in the illustrated preferred embodiment, are arranged between the circuit board 5 and the upper case 1. It is understood the two electrode-contacting elements 53, 54 may be other types of elastic contacting elements.

The first electrode-contacting element 53 has an end extended through the first through hole 13 on the first seat 11 to contact with a conductive contact area 33 provided on the first electrode 3, so that an electric stimulating signal generated by a control circuit on the circuit board 5 is sent to the first electrode 3 via the first electrode-contacting element 53.

Similarly, the second electrode-contacting element 54 has an end extended through the second through hole 16 on the second seat 14 to contact with a conductive contact area 42 provided on the second electrode 4, so that another electric stimulating signal generated by the control circuit on the circuit board 5 is sent to the second electrode 4 via the second electrode-contacting element 54.

Moreover, the circuit board 5 is provided at predetermined positions near an outer side thereof with a plurality of switch elements for controlling various movements of the electric stimulator of the present invention. For example, the switch elements may include a power switch 61 that may also function as a regulating switch to regulate an intensity of the electric stimulating signals output by the electric stimulator. A plastic push button 62 may be connected to an outer side of the power switch 61 to protectively cover the same. The switch elements may also include a function-selection switch 63 to switch the electric stimulator among different operating modes. Similarly, a plastic push button 64 may be connected to an outer side of the function-selection switch 63 to protectively cover the same.

A battery compartment 22 is formed in the lower case at one lateral side thereof for accommodating a battery 7 therein. Positive (+) and negative (−) electrodes of the battery 7 are electrically connected to corresponding contacts provided on the circuit board 5 via positive and negative contact plates 65, 66, respectively, arranged in the lower case 2, so that a working power is supplied from the battery 7 to the control circuit on the circuit board 5.

Finally, the battery compartment 22 of the lower case 2 is closed with a compartment cover 8 by hooking generally known retaining hooks 81 on the compartment cover 8 to the lower case 2.

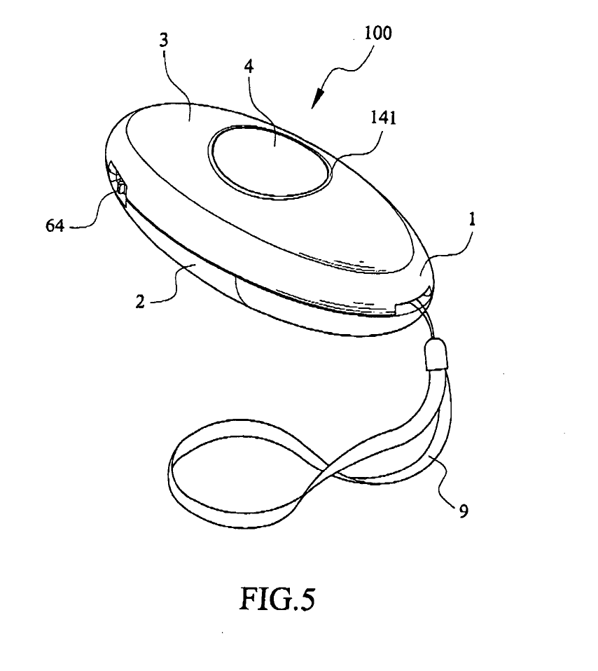

One end of the upper case 1 may be formed with a cavity having a tie bar 17 provided thereat. A loop 9 may be tied to the tie bar 17, as shown in FIG. 5, to enable convenient holding of the electric stimulator 100 at the loop 9 by a user. It is also possible to hang the loop 9 of the miniature pocket electric stimulator 100 on the user's neck or wrist.

To use the present invention, a user first turns on the power switch 63 and sets the electric stimulator to a selected operating mode or function, and then simply holds the electric stimulator with one hand. At this point, electric stimulating signals generated at the first and the second electrode 3, 4 automatically electrically stimulate points or other local areas at the hand. Of course, the electric stimulator of the present invention may also be used to electrically stimulate points or local areas on other parts of the user's body.

The electric stimulator 100 may be provided at predetermined positions on the lower case 2 with one or more indicating lights 23 in the form of light-emitting diodes (LED), as shown in FIG. 3. Or alternatively, a liquid crystal display (LCD) (not shown) may be provided on the electric stimulator at a predetermined position to display operating states and different user settings of the electric stimulator.

From the above-described preferred embodiment, it can be found the miniature pocket electric stimulator of the present invention has a modularized structure and does not require connection of output conductors thereto and application of conductive paste thereon. The present invention is therefore novel and improved as compared to conventional electric stimulators.

What is claimed is:

1. A miniature pocket electric stimulator, comprising:
a case defining an internal space;
a circuit board mounting in said internal space of said case for generating a series of electric stimulating signals;
a first seat, at a predetermined position of which a first through hole is provided;
a second seat in the form of a platform raised from a predetermined position on said first seat, said second seat being provided around a periphery with a wall portion, and at a predetermined position with a second through hole;
a first electrode having an opening corresponding to said second seat, so that said first electrode adapted to connect to said first seat; and
a second electrode being adapted to connect to said second seat via said opening on said first electrode, so that said second electrode is separated from said first electrode by said wall portion of said second seat; and
said case being provided in said internal space with a first electrode-contacting element having an end extended through said first through hole on said first seat to contact with said first electrode, and another end contacted with said circuit board, so that an electric stimulating signal generated by a control circuit on said circuit board is sent to said first electrode via said first electrode-contacting element; and a second electrode-contacting element having an end extended through said second through hole on said second seat to contact with said second electrode, and another end contacted with said circuit board, so that another electric stimulating signal generated by said control circuit on said circuit board is sent to said second electrode via said second electrode-contacting element.

2. The miniature pocket electric stimulator as claimed in claim 1, wherein said first and second electrodes are made of a plastic material, which is then electrically plated to form a conductive plated coating on an outer surface thereof.

3. The miniature pocket electric stimulator as claimed in claim 1, wherein said first and second electrodes are made of a conductive rubber material.

4. The miniature pocket electric stimulator as claimed in claim 1, wherein said first and second electrodes are made of an aluminum material.

5. The miniature pocket electric stimulator as claimed in claim 1, wherein said first and second electrodes are made of a metal material.

6. The miniature pocket electric stimulator as claimed in claim 1, wherein said first and second electrode-contacting elements are coil springs.

7. The miniature pocket electric stimulator as claimed in claim 1, wherein said first electrode comprises a conductive contact area which transmits an electric stimulating signal generated by said control circuit on said circuit board to said first electrode.

8. The miniature pocket electric stimulator as claimed in claim 1, wherein said second electrode comprises a conductive contact area which transmits another electric stimulating signal generated by the control circuit on said circuit board to said second electrode.

9. The miniature pocket electric stimulator as claimed in claim 1, wherein the stimulator comprises a power switch that may also function as a regulating switch to regulate an intensity of the electric stimulating signals output by the electric stimulator.

10. The miniature pocket electric stimulator as claimed in claim 1, wherein the stimulator comprises a function-selection switch to switch the electric stimulator among different operating mode.
